(19) United States
(12) Patent Application Publication
(10) Pub. No.: US 2022/0126102 A1
(43) Pub. Date: Apr. 28, 2022

(54) SYSTEM FOR PLANNING AND/OR CONTROLLING NEUROMODULATION

(71) Applicant: ONWARD MEDICAL B.V., Eindhoven (NL)

(72) Inventors: Robin Brouns, Eindhoven (NL); Vincent Delattre, Eindhoven (NL)

(73) Assignee: ONWARD MEDICAL B.V., Eindhoven (NL)

(21) Appl. No.: 17/511,496

(22) Filed: Oct. 26, 2021

(30) Foreign Application Priority Data
Oct. 26, 2020 (EP) 20203944.2

(51) Int. Cl. A61N 1/372; A61N 1/36

(52) U.S. Cl. CPC A61N 1/3727; A61N 1/37282; A61N 1/36189

(57) ABSTRACT

A neuromodulation system includes a medical device; a first computing device; a second computing device; and a third computing device. The system is configured to control communications by: permitting only unidirectional communication between the first computing device and the second computing device while: preventing communication between the first computing device and the third computing device, and preventing communication between the second computing device and the third computing device; permitting only unidirectional communication between the first computing device and the third computing device while: preventing communication between the first computing device and the second computing device, and preventing communication between the second computing device and the third computing device; or permitting only unidirectional communication between the second computing device and the third computing device while: preventing communication between the first computing device and the second computing device, and preventing communication between the first computing device and the third computing device.

### CROSS REFERENCE TO RELATED APPLICATIONS

The present application claims priority to European Patent Application No. EP 20203944.2, filed Oct. 26, 2020, the disclosure of which is incorporated herein by reference.

### BACKGROUND

### Technical Field

Systems and methods consistent with the present disclosure generally relate to the treatment of neurological disorders. More particularly, systems and methods consistent with the invention relate to the planning and/or controlling of neuromodulation for patients.

### Discussion of the Related Art

Neuromodulation systems typically include at least a medical device for applying neuromodulation to a patient, a controller and at least one programmer.

Medical professionals (e.g. therapists, nurses, physiotherapists, clinicians) and patients typically communicate with a medical device using an electronic programmer, in particular a clinician programmer (e.g. space time programmer) and a patient programmer respectively. Each of these programmers can be a handheld device. A clinician programmer allows a medical professional to define the particular electrical stimulation therapy to be delivered to a target area of the patient's body, while a patient programmer allows e.g. a patient or his/her relatives, to alter one or more parameters of the electrical stimulation therapy and/or start and/or stop stimulation and/or monitor properties of the neuromodulation system or a patient's health metrics (e.g. physiological data while receiving electrical stimulation therapy).

Due to multiple devices in traditional neuromodulation systems sending multiple concurrent communications, these systems are susceptible to data interference which could result in a patient failing to receive the correct neuromodulation as intended.

In view of the foregoing, it is desirable to provide neuromodulation in a manner that is not susceptible to data inference. For example, there is a need for an improved method and system to plan and control neuromodulation without data interference.

### SUMMARY

A system for planning and/or controlling neuromodulation for a patient comprises: a medical device; a first computing device configured to be in bi-directional communication with the medical device; a second computing device; a third computing device; wherein the system is configured to control communications by: permitting only unidirectional communication between the first computing device and the second computing device while: preventing communication between the first computing device and the third computing device, and preventing communication between the second computing device and the third computing device; permitting only unidirectional communication between the first computing device and the third computing device while: preventing communication between the first computing device and the second computing device, and preventing communication between the second computing device and the third computing device; or permitting only unidirectional communication between the second computing device and the third computing device while: preventing communication between the first computing device and the second computing device, and preventing communication between the first computing device and the third computing device.

A method for planning and/or controlling neuromodulation for a patient comprises: accessing, from a second computing device, an applications module of a first computing device; presenting to a user, at the second computing device, information from the applications module; transmitting data from the second computing device to the third computing device; updating neuromodulation control data at the third computing device; transferring the updated neuromodulation control data from the third computing device to the first computing device; updating installed control commands on the second computing device with the updated neuromodulation control data; sending instructions from the second computing device to the first computing device for controlling a medical device based on the updated installed control commands; and wherein a connection managing module is configured to control communications by: permitting only unidirectional communication between the first computing device and the second computing device while: preventing communication between the first computing device and the third computing device, and preventing communication between the second computing device and the third computing device; permitting only unidirectional communication between the first computing device and the third computing device while: preventing communication between the first computing device and the second computing device, and preventing communication between the second computing device and the third computing device; or permitting only unidirectional communication between the second computing device and the third computing device while: preventing communication between first computing device and the second computing device, and preventing communication between the first computing device and the third computing device.

A computer-implemented system for optimizing computer implemented tasks comprises: a memory storing instructions; and at least one processor configured to execute the instructions to: access, from a second computing device, an applications module of a first computing device; present to a user, at the second computing device, information from the applications module; transmit data from the second computing device to the third computing device; updated neuromodulation control data at the third computing device; transfer the updated neuromodulation control data from the third computing device to the first computing device; update installed control commands on the second computing device with the updated neuromodulation control data; send instructions from the second computing device to the first computing device for controlling a medical device based on the updated installed control commands; and wherein a connection managing module is configured to control communications by: permitting only unidirectional communication between the first computing device and the second computing device while: preventing communication between first computing device and the third computing device, and preventing communication between the second computing device and the third computing device; permitting only unidirectional communication between the first computing device and the third computing device while: preventing communication between first computing device and the second computing device, and preventing communication between the second computing device and the third computing device; or permitting only unidirectional communication between the second computing device and the third computing device while: preventing communication between first computing device and the second computing device, and preventing communication between the first computing device and the third computing device.

### BRIEF DESCRIPTION OF THE DRAWINGS

The accompanying drawings, which are incorporated in and constitute a part of this disclosure, illustrate various embodiments and aspects of the present invention. In the drawings:

FIG. 1 illustrates a general schematic of an embodiment of the neuromodulation system;

FIG. 2 is a flowchart of a method performed with the system depicted in FIG. 1;

FIG. 3 illustrates the schematic of FIG. 1 when unidirectional connection and/or data transfer is between a second computing device and a first computing device;

FIG. 4 illustrates the schematic of FIG. 1 when unidirectional connection and/or data transfer is between the second computing device and a third computing device;

FIG. 5 illustrates the schematic of FIG. 1 when no connection and/or data transfer is among the first computing device, the second computing device, and the third computing device;

FIG. 6 illustrates the schematic of FIG. 1 when unidirectional connection and/or data transfer is between the third computing device and the first computing device;

FIG. 7 illustrates the schematic of FIG. 1 when unidirectional connection and/or data transfer is between the third computing device and the second computing device; and

FIG. 8 illustrates the schematic of FIG. 1 when unidirectional connection and/or data transfer is between the second computing device and the first computing device.

### DETAILED DESCRIPTION

The following detailed description refers to the accompanying drawings. Wherever possible, the same reference numbers are used in the drawings and in the following description to refer to the same or similar parts. While several exemplary embodiments and features of the invention are described herein, modifications, adaptations, and other implementations are possible without departing from the spirit and scope of the invention. For example, substitutions, additions, or modifications may be made to the components illustrated in the drawings, and the exemplary methods described herein may be modified by substituting, reordering, or adding steps to the disclosed methods. Accordingly, the following detailed description does not limit the invention. Instead, the proper scope of the invention is defined by the appended claims.

Systems and methods consistent with the invention generally relate generally relate to the treatment of neurological disorders. More particularly, systems and methods consistent with the invention relate to the planning and/or controlling of neuromodulation for patients.

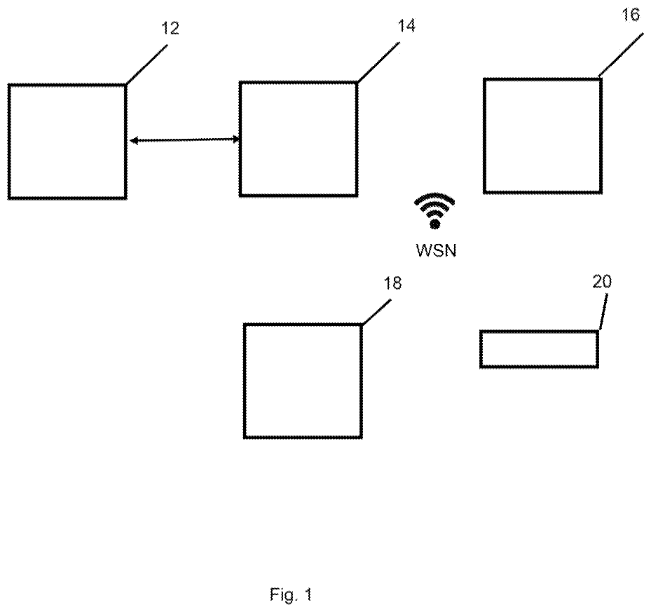

FIG. 1 depicts the components of a neuromodulation system 10 including medical device 12, first computing device 14, second computing device 18, third computing device 16, and connecting managing module 20.

Each of the components of system 10 may be physically separated from another and methods consistent with the present disclosure may be performed using one or more of the components. Even though the components may be physically separated, the components may still be communicably connected via wired or wireless technology. For example, different components of system 10, the first computing device 14, the second computing device 18, and the third computing device 16 may be connected through the Internet, a LAN (local area network), a WAN (wide area network), databases, servers, RF (radio frequency) signals, cellular technology, Ethernet, telephone, “TCP/IP” (transmission control protocol/internet protocol), and any other electronic communication format.

The medical device 12 may be a neuromodulator, in particular a neurostimulator. Also, the medical device 12 may be a battery powered device. The medical device 12 may be or may include a pulse generator 12. In some embodiments, pulse generator 12 may be an implantable pulse generator (IPG) configured to be implanted subcutaneously in a patient. The IPG may include or may be linked to one or multiple stimulation leads, each with at least one electrode. In some embodiments, pulse generator 12 may be a non-implantable pulse generator configured to be attached directly on the skin of a patient. The pulse generator can be configurable to deliver electrical stimulation via the one or more stimulation leads to one or more stimulation sites of a patient equipped with the system 10. The pulse generator can deliver such electrical stimulation in response to, or based on, commands (e.g., neuromodulation control data) received from the first computing device 14.

The first computing device 14, the second computing device 18, and the third computing device 16 may each be a general purpose computer, a personal computer, a workstation, a mainframe computer, a notebook, a global positioning device, a laptop computer, a smart phone, a personal digital assistant, a network server, and any other electronic device suitable for developing programming code.

The first computing device 14, the second computing device 18, and the third computing device 16 may include a processor, a display device, and a memory device. The first computing device 14, the second computing device 18, and the third computing device 16 may contain other components, such as components that facilitate electronic communication or user interface devices such as an input and output devices (not shown). The first computing device 14, the second computing device 18, and the third computing device 16 may include computer hardware components such as a combination of Central Processing Units (CPUs) or processors, buses, memory devices, storage units, data processors, input devices, output devices, network interface devices, and other types of components that will become apparent to those skilled in the art. The first computing device 14, the second computing device 18, and the third computing device 16 may further include application programs that may include software modules, sequences of instructions, routines, data structures, display interfaces, and other types of structures that execute operations of the present invention.

One of the hardware components in the first computing device 14, the second computing device 18, and the third computing device 16 may be a processor. The processor may be an ASIC (Application Specific Integrated Circuit) or it may be a general purpose processor. The processor may include more than one processor. For example, processors may be situated in parallel, series, or both in order to process all or part of the computer instructions that are to be processed.

One of the hardware components in the first computing device 14, the second computing device 18, and the third computing device 16 may be a memory device. Such a memory device may include all forms of computer-readable storage mediums such as non-volatile or volatile memories including, by way of example, semiconductor memory devices, such as EPROM, RAM, ROM, DRAM, EEPROM, and flash memory devices; magnetic disks such as internal hard disks and removable disks; magneto-optical disks; DVD disks, and CD-ROM disks. The memory device may be used to store program code for various application modules.

One of the hardware components in the first computing device 14, the second computing device 18, and the third computing device 16 may be a display device. Such a display device may be any conventional user interface display device. For example, the display device may include computer monitors, televisions, and LCD displays. The display device may display GUI (Graphical User Interface) which allows a user to interact with the system 10 hardware and software applications.

The first computing device 14 may be configured as an intermediate computing device 14 such as a controller 14 (i.e., a motion controller) configured to execute control software such as an applications module. In some embodiments, the controller 14 may be an implantable controller configured to be implanted subcutaneously in a patient. In some embodiments, the controller 14 may be a non-implantable controller.

The first computing device 14 may be configured to process data that is acquired e.g. from the second computing device 18, the third computing device 16 and/or the medical device 12. The first computing device 14 can (e.g., in response to acquisition or processing of such data) program the medical device 12 to deliver correct (planned) stimulation. In other words, the intermediate computing device can be configured and arranged to provide neuromodulation control data to the medical device, in particular neuromodulation control data for at last one specific task (e.g. an activity training/exercise/muscular response/autonomic system response/sphincter response/blood pressure response) and/or at least one specific neuromodulation therapy (e.g. to restore motoric function, autonomic function, blood pressure (hypotension, hypertension), bladder control, bowel control, sphincter control and/or sexual function). The content of the neuromodulation control data, or the provision of the neuromodulation control data, can depend at least partially on the information obtained from the second computing device 18 and/or the third computing device 16.

The second computing device 18 may be configured as a first user input terminal 18 such as a patient programmer 18. The patient programmer 18 may allow the user (e.g. the patient or a relative of the patient) to select a specific stimulation program and/or input therapy parameter (e.g. for a specific task or a specific medical indication) and/or to alter one or more parameters of the stimulation therapy program and/or to start and/or stop and/or ramp up and/or ramp down and/or modify and/or pause stimulation and/or to monitor properties of the neuromodulation system or at least one of patient's health metrics (e.g. physiological data while receiving electrical stimulation therapy).

In one embodiment, the second computing device 18 can be configurable to receive data from the first computing device 14 or the third computing device 16, display data, receive input from the user (e.g. patient), send it back to the first computing device 14 or the third computing device 16. Accordingly, the second computing device 16 can receive, display, process and/or resend data.

The second computing device 18 may include a display with a GUI that may offer a list of pre-installed (simplified) control commands to be selected from and/or altered by the user. In particular, by selecting and/or altering at least one of a pre-installed control commands simple handling of the system is enabled for the user.

The third computing device 16 may be configured as a second user input terminal 16 such as a clinician programmer 16. The clinician programmer 16 can be used to receive, for example, stimulation parameters, patient data, physiological data, training data etc. The clinician programmer 16 can be used for defining or altering stimulation parameters (e.g., electrode configuration, frequency, pulse width, amplitude), or allowing adjustment(s) to the stimulation. The third computing device 16 may additionally or alternatively read or monitor properties of the neuromodulation system or at least one of patient's health metrics (e.g. physiological data before, while or after receiving electrical stimulation therapy, such as blood pressure, heartrate, SpO₂, movement, number of steps, etc.). The third computing device 16 may include a space time programmer (STP). The STP can be useable to program spatial and temporal parameters of the stimulation.

In one embodiment, the third computing device 16 can be configurable to receive data from the first computing device 14 or from the second computing device 18, display data, receive input from the user (e.g. clinician or therapist), send it back to the first computing device 14 or the second computing device 18. Accordingly, the third computing device 16 can receive, display, process and/or resend data. The third computing device can be arranged such that it is configurable to receive stimulation parameters, patient data and the like, check and/or reprogram the stimulation settings and send the data back to e.g., the intermediate computing device 14.

In some embodiments, the second user input terminal 16 can comprise a display. Also, the second user input terminal 16 can comprise a user interface, in particular a graphical user interface.

In various embodiments, the first user input terminal 18 or the second user input terminal 16 can comprise a display and/or a graphical user interface. In such embodiments, the second user input terminal 16 may not include a display. The second user input terminal 16 can be remote from the controller 14 or the implantable pulse generator 12. The first user input terminal 18 can be remote from the controller 14 or the implantable pulse generator 12.

The connection managing module 20 may be embodied as a software module, in particular in the intermediate computing device 14. Additionally, or alternatively, the connection managing module 20 may be embodied as a software module in the first user input terminal 18. Additionally, or alternatively, the connection managing module 20 can be embodied in the second user input terminal 16. Alternatively, the connection managing module 20 may be embodied in another device comprised within or linked to the system. Alternatively, the at least one connection managing module 20 may be or may include a communication controller, in particular an electrical communication controller.

In some embodiments, the system 10 can comprise more than one connection managing module 20. In such embodiments, the implantable pulse generator 12 and the controller 14 can be connected. The connection between the implantable pulse generator 12 and the controller 14 can be a bidirectional connection. The connection between the implantable pulse generator 12 and the controller 14 can be a permanent and cable-bound connection. In an alternative embodiment, the connection could be a wireless connection, e.g. a telemetry connection, wireless network connection, etc. In general, also a unidirectional connection could be possible.

The first user input terminal 18 and the second user input terminal 16 can be at least temporarily connected, in particular for data transfer. Also, the first user input terminal 18 and the controller 14 can be at least temporally connected, in particular for data transfer. Also, the second user input terminal 16 and the controller 14 can be at least temporally connected, in particular for data transfer.

The connection between the first user input terminal 18 and the controller 14, the first user input terminal 18 and the second user input terminal 16, or the controller 14 and the second user input terminal 16 can be a wireless connection (e.g., a connection via a wireless network).

Alternatively, or additionally, the connection between at least one of the implanted pulse generator 12, the controller 14, the first user input terminal 18, or the second user input terminal 16 and the connection managing module 20 can be enabled by at least one of a telemetry module, Bluetooth module, or communication module. In such embodiments, the connection managing module 20 may allow only unidirectional connection or unidirectional data transfer between the controller 14 and the first user input terminal 18, the controller 14 and the second user input terminal 16, or the first user input terminal 18 and the second user input terminal 16.

The connection managing module can block bidirectional connection or data transfer between the controller 14 and the first user input terminal 18, the controller 14 and the second user input terminal 16, or the first user input terminal 18 and the second user input terminal 16.

Further, in such embodiments, the connection managing module 20 can either provide 1) connection or data transfer between the controller 14 and the first user input terminal 18 or between the controller 14 and the second user input terminal 16, or 2) connection or data transfer between the first user input terminal 18 and the second user input terminal 16. The connection managing module 20 may only provide connection or data transfer between the controller 14 and the first user input terminal 18 or the controller 14 and the second user input terminal 16 when there is no connection or data transfer between the first user input terminal 18 and the second user input terminal 16. The connection managing module 20 may block connection or data transfer between the controller 14 and the first user input terminal 18 or the controller 14 and the second user input terminal 16 when there exists a connection or data transfer between the first user input terminal 18 and the second user input terminal 16.

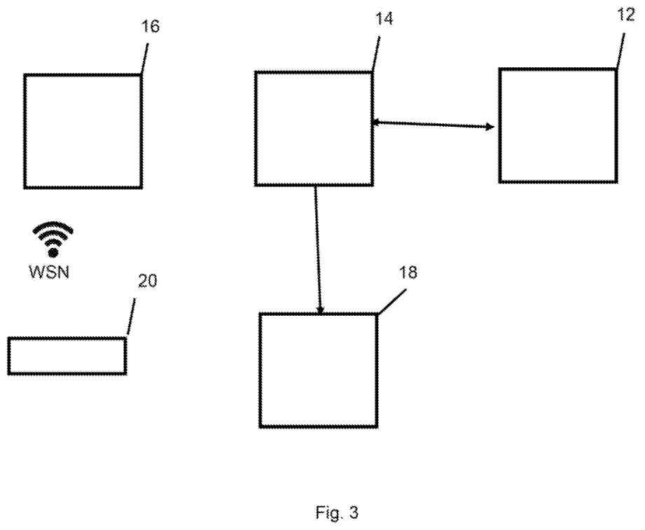

The connection managing module 20 can switch a connection or data transfer between the different components of the system 10, as seen in FIGS. 3-8.

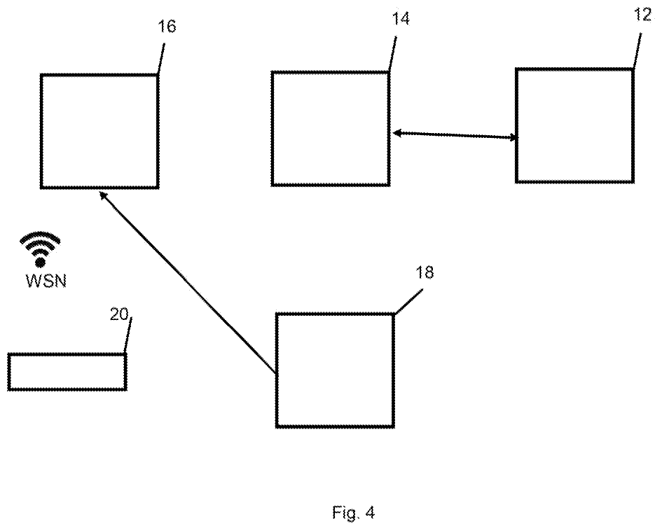

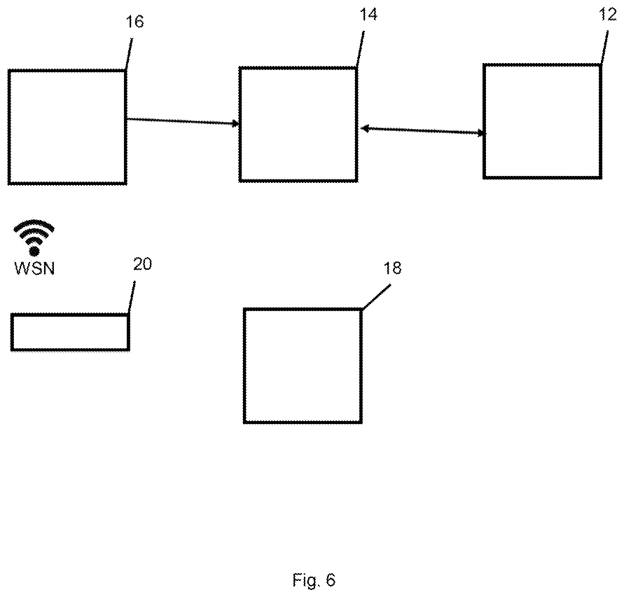

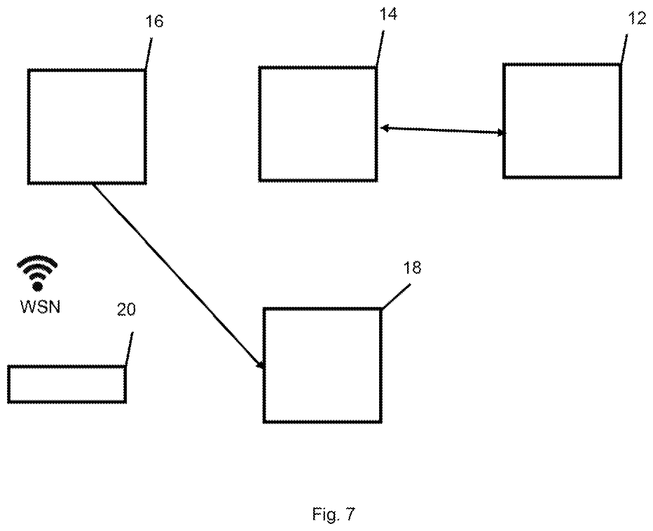

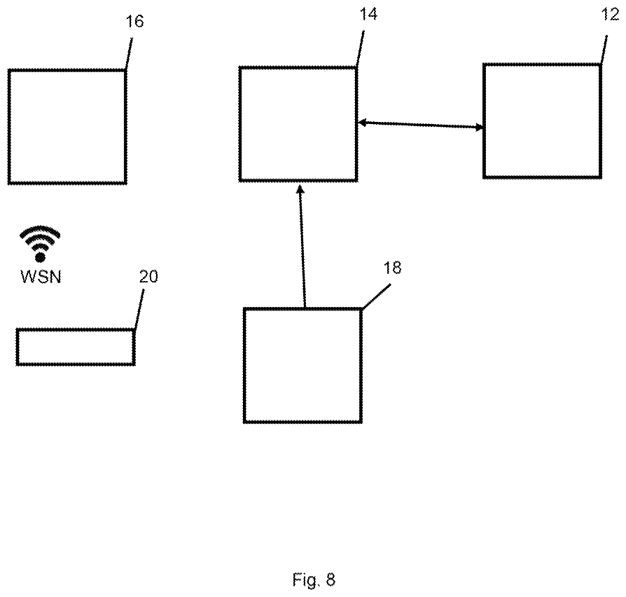

Not explicitly shown in FIG. 1 is that the system 10 enables transfer of data from the first user input terminal 18 to the second user input terminal 16 or vice versa (cf. FIGS. 4 and 7), the first user input terminal 18 to the controller 14 or vice versa (cf. FIGS. 3 and 8), the second user input terminal 16 to the controller 14 or vice versa (cf. e.g. FIG. 6) and/or the controller 14 to the medical device 12 and/or vice versa (FIGS. 3-8).

The first user input terminal 18 and the second user input terminal 16 can access or control the applications module (e.g., remotely access or control the applications module).

Not shown in FIG. 1 is that the controller 14 can present information from the applications module to a user (e.g. a patient) via the display of the first user input terminal 18. For example, input therapy parameters for section may be presented to the user. Additionally, or alternatively, the controller 14 can present information from the applications module to a user (e.g. a clinician) via the display of the second user input terminal 16. For example, stimulation parameters may be presented.

Not shown in FIG. 1 is that the user interface of the first user input terminal 18 can receive input from a user. The user input can then be processed by the first user input terminal 18 or transmitted to at least one of the controller 14 or the second user input terminal 16. For example, processed data can be transmitted to the controller 14 or the second user input terminal 16.

Also not shown in FIG. 1 is that the user interface of the second user input terminal 16 can receive input from a user. The user input can then be processed by the second user input terminal 16 or transmitted to the controller 14 or the first user input terminal 18. For example, processed data can be transmitted to the controller 14 or the first user input terminal 18.

Further not shown is that (e.g., based on the user input or the processed user input) the controller 14 can receive an indication of the input from a user and transmit the indication to the implantable pulse generator 12. In some instances, the controller can receive an indication of a selected instruction (e.g., selected from a plurality of instructions) for the implantable pulse generator 12. The controller can transmit an indication of the selected instruction to the implantable pulse generator 12.

In some such embodiments, the selected instruction can comprise at least one of a request for diagnostic data from the implantable pulse generator 12 (e.g. measured neuroelectric activity), patient data stored within the implantable pulse generator, or at least one input therapy parameter for programming into the implantable pulse generator 12. The implantable pulse generator 12 can provide the controller 14 with the diagnostic data or with patient data, or can provide stimulation based on the input therapy parameters selected.

Thus, in other words, system 10 can allow for remotely accessing or controlling a controller 14 using a first user input terminal or a second user input terminal 16. Such remote access or controlling can include providing user input to indicate a selected instruction from a plurality of instructions (e.g., using a user interface of the first user input terminal 18 or second user input terminal 16). The controller 14 can communicate the selected instruction to an implanted pulse generator 12. The system 10 can also allow only unidirectional connection or unidirectional data transfer between the controller 14 and the first user input terminal 18, the controller 14 and the second user input terminal 16, or the first user input terminal 18 and the second user input terminal 16. The system 10 can provide only connection or data transfer between the controller 14 and the first user input terminal 18 or between the controller 14 and the second user input terminal 16, when there is no connection or data transfer between the first user input terminal 18 and the second user input terminal 16.

In the context of neuromodulation, especially neurostimulation, connection managing module 20 can support reliable planning or controlling of stimulation parameters. In this manner, data interference can be prevented or reduced, supporting implementation of precise stimulation protocols. Interference of data or signals transmitted between user input terminal 18 and user input terminal 16, or between user input terminal 18 and intermediate computing device 14 can reduced or prevented by avoiding bidirectional connection or data transfer between these units. In other words, unidirectional communication channels can be provided provided. In other words, one device, module or terminal may send data or instructions in one direction to another device, module or terminal. Therefore, the disclosed embodiments can enable controlled and secure transmission of data or signals during operation of the system. Further, the disclosed embodiments can enable encryption of such data or signals. Further, the disclosed embodiments can reduce energy consumption of the system and prolonged battery life through unidirectional data transfer or selective temporal connections.

Data interference may occur when transmissions are sent from a transmitter (device, module or terminal) to a receiver (other device, module or terminal) and when transmissions are simultaneously sent from said receiver (which is then also a transmitter) to the transmitter (which is then also a receiver). Also, data interference may occur within a system when transmissions are sent from a transmitter (device, module or terminal) to a receiver (other device, module or terminal) and when transmissions are simultaneously sent from another transmitter to another or the same receiver.

Data interference may alternatively or additionally occur when data are stored in at least one component of the system 10 (e.g. intermediate computing device 14, first user input terminal 18, second user input terminal 16, medical device 12 or controller 20). In particular, the data stored on the at least one unit may be newer or older than data stored in another, connected device (other element of the system). In particular, interference of data may then be prevented or reduced by adapting the data stored in both units to the newer version or by avoiding simultaneous data transfer between each of these units with a third unit.

In particular, the intermediate computing device 14 can be configurable to perform sanity-checks on commands received from the first user input terminal 18 (or from the second user input terminal 16). Such sanity-checks can prevent the first user input terminal 18 (or the second user input terminal 16) from sending invalid commands directly into the medical device. In particular, the intermediate computing device 14 may comprise or use a security system that monitors or controls incoming or out coming signal traffic based on predetermined security rules, such as an unauthorized access block method, e.g. ‘a firewall’. This may, for instance, enable secure data transfer.

The first or the second user input terminal 18, 16 may be configurable to send and receive data to or from a remote database. The first or the second user input terminal 18, 16 may comprise a display or a graphical user interface. The first and the second user input terminal 18, 16 can be embodied as applications installed on a mobile device that communicate with the intermediate computing device/controller 14. Such applications can be used by the patient and the treating physician or physiotherapist, respectively, to provide inputs to the intermediate computing device/controller 14. The first user input terminal 18 or the second user input terminal 16 can allow adjustment of the stimulation parameters of a task, while the task (i.e. a stimulation program aimed at addressing a specific neurological function, such as locomotion) is running. This enables the user to tune the stimulation without having to start and stop the task, which would be very cumbersome and clinically undesired at the start of the rehabilitation training (e.g., when all stimulation partitures are being developed and tuned).

In some embodiments, the system 10 can further comprise a telemetry module or Bluetooth module or communication module or wireless network enabling connection between the medical device 12, the intermediate computing device 14, the first user input module 18 and/or the second user input module 16. This may advantageously enable wireless or remote control or programming of the stimulation. For instance, the second user input terminal 16 and its user can be remote from the first user input terminal 18.

In some embodiments, the connection between the medical device 12 and the intermediate computing device can be a bidirectional connection. In particular, the connection may be a wireless or cable-bound connection. In some instances, this connection may be a constant connection. A constant connection may enable continuous data transfer between the medical device and the intermediate computing device. For instance, the medical device can provide the intermediate computing device with patient data (sensed by a linked sensor), while the intermediate computing device provides the medical device with neuromodulation control data. This may be advantageous for settings where the stimulation program provided by the intermediate computing device is being (or will be) adapted to data provided by the medical device (e.g. patient data, in particular patient-feedback data).

Further, it is generally possible that the first user input terminal 18 or the second user input terminal 16 or the intermediate computing device 14 are connected by a wireless connection. This has the advantage that the first user input terminal 18 or the second user input terminal 16 can be remote from the intermediate computing device 14. The wireless connection may be any remote telemetry techniques known in the art.

In some embodiments, the first user input terminal 18 or the second user input terminal 16 can be remote from the intermediate computing device 14 or the medical device. This has the advantage that the user of the first user input terminal 18 can be remote from the user of the second user input terminal 16, and the user of the first user input terminal 18 or the user of the second user input terminal 16 can be remote from the intermediate computing device 14 or the medical device. For instance, a patient equipped with the first user input terminal 18, the medical device, and the intermediate computing device 14 can be at home and thus remote from the second user input terminal 16 (which may be e.g. placed in a hospital or laboratory).

In some embodiments, the system can be configured and arranged for transferring data from the first user input terminal 18 to the second user input terminal 16 or vice versa, the first user input terminal 18 to the intermediate computing device 14 or vice versa, the second user input terminal 16 to the intermediate computing device 14 or vice versa, or the intermediate computing device 14 to the medical device or vice versa. In particular, this may enable selective data transfer between respective transmitting devices/terminals or receiving devices/terminals without transferring data to non-targeted devices/terminals.

In some embodiments, the intermediate computing device 14 comprises an applications module, wherein the first user input terminal 18 or the second user input terminal 16 are configured and arranged for accessing or controlling the applications module (e.g., remotely accessing or controlling the applications module). In particular, the intermediate computing device 14 may present information from the applications module to a user (e.g. via a display on the first user input terminal 18 or second user input terminal 16). Such information can include a plurality of instructions for the medical device 12. In particular, remote access and control of the applications module may enable user-friendly and uncomplicated application of the system 10.

In some embodiments, the first user input terminal 18 or the second user input terminal 16 can comprise a display. The intermediate computing device 14 can presents information from the applications module to a user via the display of the first user input terminal 18 or the second user input terminal 16. This may be advantageous by enabling user-friendly application of the system (e.g. by clearly arranging on the display the patient data, the programming options, or the instructions to be selected). Thus, such embodiments can improve user understanding and handling of the system 10.

In some embodiments, the first user input terminal 18 or the second user input terminal 16 can comprise a user interface configured and arranged for receiving input from a user. In particular, the user interface may be a graphical user interface, or the display may present a graphical user interface that enables remote control of features of the intermediate computing device 14. Such embodiments may enable easy and user-friendly application of the system 10.

Also, the intermediate computing device 14 may be configured and arranged for receiving an indication of an input from a user, especially an indication of a selected instruction from a plurality of instructions for the medical device, and transmit the selected instruction to the medical device. In other words, the user input terminal may provide a plurality of possible instructions for instruction of the medical device to a user, and the user can select desired instructions which are transmitted to the intermediate computing device 14.

In particular, the selected instruction may include at least a request for diagnostic data from the medical device 12, patient data stored in the medical device 12 or an input therapy parameter for programming into the implantable medical device 12. For instance, diagnostic data may be or may comprise neurological signals from the patient (e.g. sensed by a connected electrode), patient data may comprise physiological and/or pathological data, input therapy parameter may comprise starting and/or stopping and/or ramping up and/or ramping town stimulation for a task and/or configuration of stimulation parameters (electrode configuration, pulse width, amplitude, frequency, electrode configuration). In other words, a user may select at least one instruction, and the medical device 12 operates according to the selected instruction, after the selected instructions is transmitted from the intermediate computing device 14 to the medical device 12.

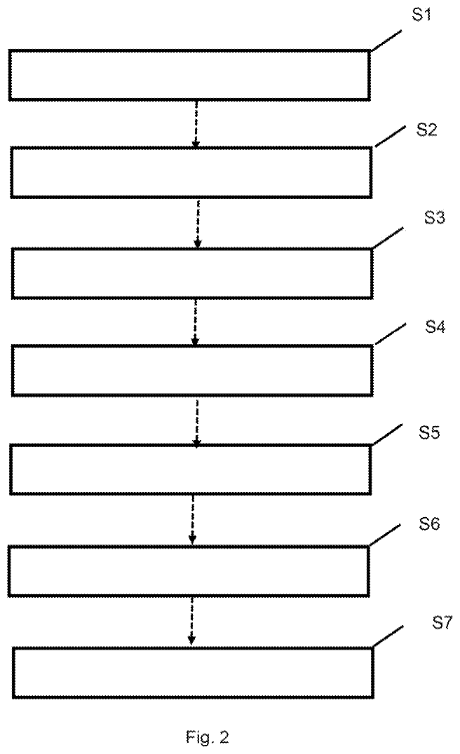

FIG. 2 is a flowchart of a method that can be performed on system 10. The method comprises at least the steps S1-S7.

A first step S1 can include accessing, from a second computing device 18, an applications module of a first computing device 14. Such accessing can be done remotely. Data concerning the implantable pulse generator 12 or concerning the controller 14 can be accessed. In this way, the applications module and (indirectly) the implantable pulse generator 12 can be accessed.

In step S1, the controller 14 can be connected to the first user input terminal 18 and data are transferred unidirectionally from the controller 14 to the first user input terminal 18, cf. FIG. 3.

A second step S2 can include presenting to a user, at the second computing device 18, information from the applications module. This presentation can be done, for example, using the display of the second computing device 18.

A third step S3 can include transmitting data from the second computing device 18 to the third computing device 16. In step S3, a connection between the first user input terminal 18 and the second user input terminal 16 or a unidirectional data transfer from the first user input terminal 18 to the second user input terminal 16 can be enabled. A connection or data transfer between the first user input terminal 18 and the controller 14 and the second user input terminal 16 and the controller 14 may not be enabled (or may be disabled), cf. FIG. 4. Accordingly, incidental overwriting of neuromodulation control data can be avoided.

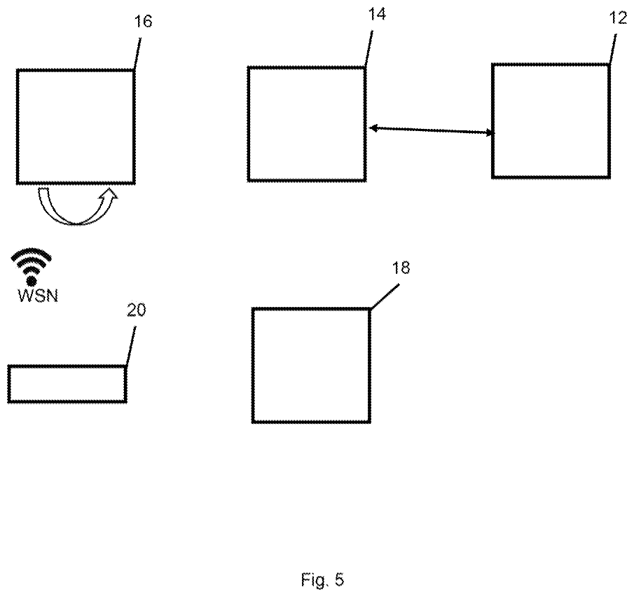

A fourth step S4 can include updating neuromodulation control data at the third computing device 16. This updated neuromodulation control data may be based on the data previously transmitted from the second computing device 18. In step S4, a connection or data transfer between the second user input terminal 16 and the first user input terminal 18 or the controller 14 may not be enabled (or may be disabled), cf. FIG. 5. Accordingly, incidental control commands or overwriting of neuromodulation control data can be avoided.

A fifth step S5 can include transferring the updated neuromodulation control data from the third computing device 16 to the first computing device 14. In step S5, a connection between the second user input terminal 16 and the controller 14 or unidirectional data transfer from the second user input terminal 16 to the controller 14 can be enabled. A connection or data transfer between the first user input terminal 18 and the controller 14 and the second user input terminal 16 and the first user input terminal 18 may not be enabled (or may be disabled), cf. FIG. 6. Accordingly, incidental control commands or overwriting of neuromodulation control data provided from the first user input terminal 18 can be avoided.

A sixth step S6 can include updating installed control commands (e.g., input therapy parameters) on the second computing device with the updated neuromodulation control data. In step S6, a connection between the second user input terminal 16 and the first user input terminal 18 or unidirectional data transfer from the second user input terminal 16 to the first user input terminal 18 can be enabled. A connection or data transfer between the first user input terminal 18 and the controller 14 and the second user input terminal 16 and the controller 14 may not be enabled (or may be disabled), cf. FIG. 7. Accordingly, interference of transferred data with neuromodulation control commands stored in the intermediate computing device 14 can be avoided.

A seventh step S7 can include sending instructions from the second computing device to the first computing device for controlling a medical device based on the updated installed control commands (e.g., input therapy parameters). In step S7, connection between the first user input terminal 18 and the controller 14 or unidirectional data transfer from the first user input terminal 18 to controller 14 can be enabled. A connection or data transfer between the second user input terminal 16 and the controller 14 and the second user input terminal 16 and the first user input terminal 18 may not be enabled (or may be disabled), cf. FIG. 8. Accordingly, data interference can be avoided. Also, as pre-installed control commands are presented to the user, such embodiments can simplify handling of the system by the user (e.g. a patient) and improve the usability and safety of the system.

Thus, certain embodiments allow only unidirectional connection or unidirectional data transfer between the controller 14 and the first user input terminal 18, between the controller 14 and the second user input terminal 16, or between the first user input terminal 18 and the second user input terminal 16. Further, such embodiments may only provide a connection or data transfer between the controller 14 and the first user input terminal 18, or between the controller 14 and the second user input terminal 16, when there is no connection or data transfer between the first user input terminal 18 and the second user input terminal 16.

Disclosed embodiments can prevent or reduce interference of data or signals transmitted among user input terminal 18, user input terminal 16, and the intermediate computing device 14. Therefore, the disclosed embodiments can support controlled and secure transmission of data or signals during system operation (e.g., continuously or at all times during system operation). Further, the disclosed embodiments can support encryption of data or signals. Also, the disclosed embodiments may save energy or power and therefore prolong battery life.

FIG. 3 shows the system 10 when unidirectional connection or data transfer is between a second computing device 18 and a first computing device 14. In such embodiments, a unidirectional connection between the controller 14 and the first user input terminal 18 or unidirectional data transfer from the controller 14 to the first user input terminal 18 is enabled. While the connection is enabled, the application module of the controller 14 can be remotely accessed by means of the first user input terminal 18 and data can be retrieved from the medical device 12.

FIG. 4 shows the system 10 when a unidirectional connection or data transfer is between the second computing device 18 and a third computing device 16. In such embodiments, the unidirectional connection between the first user input terminal 18 and the second user input terminal 16 or the unidirectional data transfer from the first user input terminal 18 to the second user input terminal is enabled. Connection or data transfer between the first user input terminal 18 and the controller 14 and the second user input terminal 16 and the controller may not be enabled (or may be disabled). The enabled connection allows data to be transmitted from the first user input terminal 18 to the second user input terminal 16.

FIG. 5 shows the system 10 when no connection or data transfer is among the first computing device 14, the second computing device 18, and the third computing device 16. No connection or data transfer between the second user input terminal 16 and the first user input terminal 18, or between the second user input terminal 16 and the controller 14, is enabled while neuromodulation control data or stimulation parameters are updated on the second user input terminal 16.

FIG. 6 shows the system 10 when unidirectional connection or data transfer is between the third computing device 16 and the first computing device 14. In this embodiment, unidirectional connection between the second user input terminal 16 and the controller 14, or unidirectional data transfer from the second user input terminal 16 to the controller 14, is enabled. Connection or data transfer between the first user input terminal 18 and the controller 14 and the second user input terminal 16 and the first user input terminal 18 may not be enabled (or may be disabled). Through the enabled connection, the controller 14 can be provided with updated neuromodulation control data.

FIG. 7 shows the system 10 when unidirectional connection or data transfer is between the third computing device 16 and the second computing device 18. In this embodiment, unidirectional connection between the second user input terminal 16 and the first user input terminal 18, or unidirectional data transfer from the second user input terminal 16 to the first user input terminal 18, is enabled. Connection or data transfer between the first user input terminal 18 and the controller 14 and the second user input terminal 16 and the controller 14 may not be enabled (or may be disabled). The enabled connection can enable updating installed control commands (input therapy parameters) on the first user input terminal 18 by means of updated neuromodulation control data provided by the second user input terminal 16.

FIG. 8 shows the system 10 when unidirectional connection or data transfer is between the second computing device 18 and the first computing device 14. In this embodiment, connection between the first user input terminal 18 and the controller 14, or unidirectional data transfer from the first user input terminal 18 to controller 14, is enabled. Connection or data transfer between the second user input terminal 16 and the controller 14 and the second user input terminal 16 and the first user input terminal 18 may not be enabled (or may be disabled). The enabled connection can enable programming or controlling the controller 14 by means of updated installed control commands (input therapy parameters).

The foregoing description has been presented for purposes of illustration. It is not exhaustive and does not limit the invention to the precise forms or embodiments disclosed. Modifications and adaptations of the invention will be apparent to those skilled in the art from consideration of the specification and practice of the disclosed embodiments of the invention. In this specification, the term “or” is used in the inclusive sense, unless context indicates otherwise.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for planning and/or controlling neuromodulation for a patient comprising:
a medical device;
a first computing device configured to be in bi-directional communication with the medical device;
a second computing device;
a third computing device;
wherein the system is configured to control communications by:
permitting only unidirectional communication between the first computing device and the second computing device while:
preventing communication between the first computing device and the third computing device, and
preventing communication between the second computing device and the third computing device;
permitting only unidirectional communication between the first computing device and the third computing device while:
preventing communication between the first computing device and the second computing device, and
preventing communication between the second computing device and the third computing device; or
permitting only unidirectional communication between the second computing device and the third computing device while:
preventing communication between the first computing device and the second computing device, and
preventing communication between the first computing device and the third computing device.

2. The system of claim 1, wherein the medical device, the first computing device, the second computing device, and the third computing device are configured to communicate with each other using wireless communications.

3. The system of claim 1, wherein the medical device comprises a neuromodulator and/or a neurostimulator.

4. The system of claim 1, wherein the first computing device comprises a controller configured to control operation of the medical device.

5. The system of claim 1, wherein the second computing device comprises a programable device configured to allow a user to: (i) select a specific stimulation program and/or a specific input therapy parameter, (ii) alter a selected stimulation program, and/or (iii) control when and how a selected stimulation program is executed.

6. The system of claim 1, wherein the third computing device comprises a programable device configured to allow a user to: (i) review at least one of stimulation data or patient data associated with the medical device, and/or (ii) adjust stimulation provided by the medical device.

7. The system of claim 1, wherein the second computing device and the third computing device each include a display for presenting information.

8. The system of claim 1, wherein the second computing device and the third computing device each include a user interface for receiving input.

9. A method for planning and/or controlling neuromodulation for a patient comprising:
accessing, from a second computing device, an applications module of a first computing device;
presenting to a user, at the second computing device, information from the applications module;
transmitting data from the second computing device to the third computing device;
updating neuromodulation control data at the third computing device;
transferring the updated neuromodulation control data from the third computing device to the first computing device;
updating installed control commands on the second computing device with the updated neuromodulation control data;
sending instructions from the second computing device to the first computing device for controlling a medical device based on the updated installed control commands; and
wherein a connection managing module is configured to control communications by:
permitting only unidirectional communication between the first computing device and the second computing device while:
preventing communication between the first computing device and the third computing device, and
preventing communication between the second computing device and the third computing device;
permitting only unidirectional communication between the first computing device and the third computing device while:
preventing communication between the first computing device and the second computing device, and
preventing communication between the second computing device and the third computing device; or
permitting only unidirectional communication between the second computing device and the third computing device while:
preventing communication between first computing device and the second computing device, and
preventing communication between the first computing device and the third computing device.

10. The method of claim 9, wherein the medical device, the first computing device, the second computing device, and the third computing device communicate with each other using wireless communications.

11. The method of claim 9, wherein the medical device comprises a neuromodulator and/or a neurostimulator.

12. The method of claim 9, wherein the first computing device comprises a controller configured to control operation of the medical device.

13. The method of claim 9, wherein the second computing device comprises a programable device configured to allow to: (i) select a specific stimulation program and/or a specific input therapy parameter, (ii) alter a selected stimulation program, and/or (iii) control when and how a selected stimulation program is executed.

14. The method of claim 9, wherein the third computing device comprises a programable device configured to allow a user to: (i) review at least one of stimulation data or patient data associated with the medical device, and/or (ii) adjust stimulation provided by the medical device.

15. The method of claim 9, wherein the method further comprises:
presenting information to a patient and a healthcare provider respectively using displays included in each of the second computing device and the third computing device.

16. The method of claim 9, wherein the method further comprises:
receiving input from a patient and a healthcare provider respectively using user interfaces included in each of the second computing device and the third computing device each.

17. A computer-implemented system for optimizing computer implemented tasks comprising:
a memory storing instructions; and
at least one processor configured to execute the instructions to:
access, from a second computing device, an applications module of a first computing device;
present to a user, at the second computing device, information from the applications module;
transmit data from the second computing device to the third computing device;
updated neuromodulation control data at the third computing device;
transfer the updated neuromodulation control data from the third computing device to the first computing device;
update installed control commands on the second computing device with the updated neuromodulation control data;
send instructions from the second computing device to the first computing device for controlling a medical device based on the updated installed control commands; and
wherein a connection managing module is configured to control communications by:
permitting only unidirectional communication between the first computing device and the second computing device while:
preventing communication between first computing device and the third computing device, and
preventing communication between the second computing device and the third computing device;
permitting only unidirectional communication between the first computing device and the third computing device while:
preventing communication between first computing device and the second computing device, and
preventing communication between the second computing device and the third computing device; or
permitting only unidirectional communication between the second computing device and the third computing device while:
preventing communication between first computing device and the second computing device, and
preventing communication between the first computing device and the third computing device.
